(19) United States
(12) Patent Application Publication
(10) Pub. No.: US 2023/0263948 A1
(43) Pub. Date: Aug. 24, 2023

(54) DIALYSIS SYSTEMS

(71) Applicant: Quanta Dialysis Technologies Limited, Warwick (GB)

(72) Inventor: Mark WALLACE, Warwick (GB)

(73) Assignee: Quanta Dialysis Technologies Limited, Warwick (GB)

(21) Appl. No.: 18/015,030

(22) PCT Filed: Jul. 5, 2021
(86) PCT No.: PCT/GB2021/051705

§ 371 Date: Jan. 6, 2023

(30) Foreign Application Priority Data
Jul. 6, 2020 (GB) 2010352.9

(51) Int. Cl. A61M 1/16

(52) U.S. Cl. CPC A61M 1/1686; A61M 1/1688; A61M 1/1603

(57) ABSTRACT

A dialysis system comprising a dialysis machine (100) having a main body portion, a water purification system, the water purification system being separate to the dialysis machine, and a liquid sanitizer (200). The liquid sanitizer (200) is provided within the main body portion of the dialysis machine. The liquid sanitizer (200) is fluidly connected between the dialysis machine (100) and the water purification system. The liquid sanitizer (200) has a heater (240) arranged to heat a volume of liquid, a temperature sensor arranged to sense the temperature of the volume of liquid and a liquid sanitizer controller (250). The dialysis system defines a first closed fluid circuit comprising the dialysis machine and the liquid sanitizer and a second closed fluid circuit comprising the water purification system, the dialysis machine and the liquid sanitizer. The liquid sanitizer (200) is configured to effect sanitization of the first closed fluid circuit and the second closed fluid circuit. A method of heat sanitization of a dialysis system.

### TECHNICAL FIELD

The present disclosure relates to a dialysis system. Particularly, but not exclusively, the disclosure relates to heat sanitization of a dialysis machine and a water purification system for use with a dialysis machine. Aspects of the invention relate to dialysis system, and a method of heat sanitization of a dialysis water circuit.

### BACKGROUND

Dialysis is a treatment which replaces the renal function of removing excess fluid and waste products, such as potassium and urea, from blood. The treatment is either employed when renal function has deteriorated to an extent that uremic syndrome becomes a threat to the body's physiology (acute renal failure) or, when a longstanding renal condition impairs the performance of the kidneys (chronic renal failure).

There are two major types of dialysis, namely hemodialysis and peritoneal dialysis. In hemodialysis, the patient's blood is removed from the body by an arterial line and treated by a dialysis machine before being returned to the patient's body by a venous line. The machine passes the blood through a dialyser containing tubes formed from a semi-permeable membrane. On the exterior of the semi-permeable membrane is a dialysis fluid. The semi-permeable membrane filters the waste products and excess fluid from the blood into the dialysis fluid. The membrane allows the waste and a controlled volume of fluid to permeate into the dialysis fluid whilst preventing the loss of larger more desirable molecules, like blood cells and certain proteins and polypeptides.

The correction of uremic acidosis of the blood is achieved by use of a bicarbonate buffer. The bicarbonate buffer also allows the correction of the blood bicarbonate level. The dialysate fluid consists of a sterilized solution of mineral ions. These ions are contained within an acid buffer which is mixed with the purified water and bicarbonate base prior to delivery to the dialyser.

Production of dialysis fluid is described in the applicant's own applications, WO2010/146342, WO2010/146344, WO2014/155121 and WO2016/120415 the entire contents of each are expressly incorporated herein by reference.

In simple terms, dialysis water is mixed with the bicarbonate buffer and the acid buffer to create a dialysis fluid for performing dialysis across a semi-permeable membrane in a dialyzer. Dialysis water is defined by the standard ISO 23500-3:2019. Dialysis fluid is defined by the standard ISO 23500-5:2019. The dialysis water may be generated by a water purification system which purifies domestic tap water by passing it through a prefiltration stage and then a reverse osmosis (RO) machine. Once supplied to the dialysis machine the dialysis water is heated to the correct temperature for treatment and is provided to the dialysis fluid mixing and pumping cassette where the acid and bicarbonate solutions are added to create dialysis fluid.

Patients being treated for a renal condition are typically required to either attend a medical facility, either in an acute setting, for example an intensive care ward or in a chronic setting, for example a dialysis ward or dialysis center. Some patient's requiring treatment for chronic conditions may be able to conduct dialysis at home.

Given the varied treatment settings, there is also a variation in the availability of dialysis water. For example a hospital dialysis ward may have access to a hospital water ring main, where dialysis water is provided from a hospital plant room. This may differ from an acute setting, such as an intensive care unit, where there is no provision of a hospital ring main. This may also differ from home use, where the only plentiful source of water is through the domestic tap. Given the varied treatment settings for dialysis, some may require the dialysis machine alone, whereas others may require both the dialysis machine and the RO machine.

Typically dialysis machines and RO machines need to be sanitized between uses and maintained in a sanitized condition. Conventionally, known dialysis machines are sanitized either through heat disinfection or chemical disinfection to ensure the microbiological quality of the supplied dialysis water and dialysis fluid is kept below acceptable levels as defined by the standard ISO 23500-5:2019. RO machines are sanitized through either through heat disinfection or chemical disinfection to ensure the microbiological quality of the supplied dialysis water is kept below acceptable levels as defined by the standard ISO 23500-3:2019. Water treatment equipment is governed by the standard ISO 23500-2:2019, and dialysis machines specifically by the standard IEC 60601-2-16:2019.

Such heat sanitization processes may be achieved by means of the A0 method which uses a knowledge of the lethality to microbiological contaminants of the particular process at different temperatures to assess the overall lethality to microbiological contaminants of the cycle and express this as the equivalent exposure time at a specified temperature. An example of the application of the A0 method to a dialysis machine is described in WO2015/185920.

Previous heat sanitizing systems for RO machines, such as the one disclosed in WO2018/202321, use a combination of a heating element located in the RO machine and chemical disinfectant in order to sanitize RO machines. This sanitization system only cleans the RO machine and not the dialysis machine attached to it.

RO machines and dialysis machines have independent sanitization routines. As such, the RO and dialysis machine must be sanitized independently. Often the devices must be disconnected from each other prior to the sanitization cycle. There is still the potential for bacteria to collect and to develop into biofilm in the fluid lines that connect the RO and dialysis machine as a result.

Known dialysis and RO systems are provided as independent systems with no shared functionality of control between the RO and dialysis machine when they are fluidly connected.

### SUMMARY OF THE INVENTION

In accordance with an aspect of the present invention, there is provided a dialysis machine having a main body portion; a water purification system, the water purification system being separate to the dialysis machine; and a liquid sanitizer; wherein the liquid sanitizer is provided within the main body portion of the dialysis machine; wherein the liquid sanitizer is fluidly connected between the dialysis machine and the water purification system,

the liquid sanitizer having a heater arranged to heat a volume of liquid, a temperature sensor arranged to sense the temperature of the volume of liquid and a liquid sanitizer controller, the dialysis system defining a first closed fluid circuit comprising the dialysis machine and the liquid sanitizer and a second closed fluid circuit comprising the water purification system, the dialysis machine and the liquid sanitizer, and wherein the liquid sanitizer is configured to effect sanitization of the first closed fluid circuit and the second closed fluid circuit.

The liquid sanitizer heater is sized for heating dialysis water at 37 degrees Celsius from a typical inlet temperature of 10 degrees Celsius and at the typical flow rate of 500 ml/min in a dialysis treatment mode. This allows the dialysis machine to produce a continuous supply of dialysis fluid to be sent to a dialyser during treatment.

In liquid sanitization mode, the liquid sanitizer heater is required to heat dialysis water to approximately 80 to 85 degrees Celsius. Further, in liquid sanitization mode, the liquid sanitizer heater is required to maintain the heated dialysis water at approximately 80 to 85 degrees Celsius. In liquid sanitization mode a finite quantity of dialysis water is circulation around a closed fluid circuit of the dialysis machine at approximately 400 ml/min.

Since in liquid sanitization mode the sanitizing liquid is circulated around a closed fluid circuit and not used to prepare a continuous supply of dialysis fluid, the volume of dialysis water that must be heated is far smaller than in treatment mode and it is also increasing in temperature as it circulates around the closed fluid circuit. The liquid sanitizer heater therefore has spare capacity to effect further fluid heating.

The present invention takes advantage of that spare capacity to extend the closed fluid circuit to optionally further include the water purification system, and thereby increase the fluid volume of the closed fluid circuit. The present invention also takes advantage of that spare capacity to account for any additional heat loss experienced by circulating the heated fluid around the extended closed fluid circuit including the water purification system. The present invention further minimises the time between treatments which is necessary to sanitize the dialysis machine and the water purification system and the operator effort required therefore.

Since the liquid sanitizer is provided within the dialysis machine, it is not necessary for the water purification system to have a heater, thus the water purification system can be smaller, simpler, and inherently more reliable.

The liquid sanitizer controller may be controlled by a dialysis machine controller. The present invention allows for central control of the liquid sanitization of both the dialysis machine and the separate water purification system. This supports the ease of use of the dialysis system.

The dialysis machine may be provided with a graphical user interface, wherein the graphical user interface can provide instructions to the dialysis machine controller.

The water purification system may comprise a reverse osmosis machine.

The reverse osmosis machine may have a controller.

The liquid sanitizer controller may be configured to determine a time-temperature value for the volume of liquid periodically once a threshold temperature has been exceeded and calculate a cumulative time-temperature value for the first closed fluid circuit and the second closed fluid circuit.

The liquid sanitizer temperature sensor may be an inlet water temperature sensor arranged on a tank inlet adjacent a liquid sanitizer tank.

A liquid sanitizer outlet valve may be positioned adjacent a liquid sanitizer outlet to control the flow via either the liquid sanitizer outlet or a liquid sanitizer return line.

The RO machine controller may be controlled by the dialysis machine controller. Typically, whilst the dialysis machine is positioned at the optimum ergonomic height for the user, liquid sanitizers are often less accessible. Therefore control of the RO machine via the dialysis machine offers a better end-user experience.

In accordance with a further aspect of the present invention there is provided a method of heat sanitization of a dialysis system, the method comprising the steps of:

providing a dialysis machine, a water purification system, and a liquid sanitizer, the liquid sanitizer being fluidly connected between the dialysis machine and the water purification system,

the liquid sanitizer having a heater arranged to heat a volume of liquid and a temperature sensor arranged to sense the temperature of the volume of liquid, heating the volume of liquid from an initial temperature to exceed a threshold temperature, maintaining the volume of water above the threshold temperature, and circulating the volume of liquid through a first closed fluid circuit comprising the dialysis machine and the liquid sanitizer and circulating the volume of liquid through a second closed fluid circuit comprising the water purification system, the dialysis machine and the liquid sanitizer to effect a sanitizing dose in the first fluid circuit and the second fluid circuit.

The steps of circulating the volume of liquid through the first closed fluid circuit and circulating the volume of liquid through the second closed fluid circuit may be terminated in a sequential fashion.

The method may comprise the further steps of determining a time-temperature value for the volume of liquid periodically once the threshold temperature has been exceeded and calculating a cumulative time-temperature value based upon the determined time-temperature value.

The cumulative time-temperature value may be calculated according to
A0 is the A value when z is 10° C.;

$${A}_{0} = Σ10 {e}^{\frac{T - 80}{z}} dt$$

t is the chosen time interval, in seconds;
and is the temperature in the load in ° C.

The A0 value for the first closed fluid circuit may be equal to A0₁ and the A0 value for the second closed fluid circuit may be equal to A0₂, where A0₂ is greater than A0₁.

The step of circulating the volume of liquid through a second closed fluid circuit may include using a water purification system pump.

The method may comprise the further step of providing an output signal once the cumulative time-temperature value has reached a level indicative of a sanitizing dose in both the first fluid circuit and the second closed fluid circuit.

The method may comprise the further step of ceasing circulation through the second closed fluid circuit once the cumulative time-temperature value equals a target cumulative time-temperature value.

The method may comprise the further step of setting at least one of the threshold temperature, the upper temperature or the target cumulative time-temperature value.

### BRIEF DESCRIPTION OF THE DRAWINGS

One or more of the embodiments of the invention can now be described, by way of example only, with reference to the accompanying drawings, in which:

FIG. 1 is a schematic view of a system comprising of a reverse osmosis machine connected to a dialysis machine; and

FIG. 2 is a schematic of the system shown in FIG. 1.

### DETAILED DESCRIPTION

A dialysis system liquid sanitization system 10 comprises a dialysis machine 100, a water purification system and a liquid sanitizer 200 fluidly connected there between. The water purification system is separate to the dialysis machine 100. In the specific example, the water purification system comprises a reverse osmosis (RO) machine 300.

The dialysis machine 100 has a main body 112 with a door 114 hinged to a forward-facing side of the main body 112. The door 114 has a graphical user interface. The graphical user interface is a Liquid Crystal Display (LCD) unit 116 disposed on an outward-facing door surface and a door platen disposed on an inward-facing surface. The LCD unit 116 is touch sensitive allowing user inputs to control the dialysis machine 100 and liquid sanitization system 10 as will be described in more detail below. In alternative embodiments user input can be provided using buttons, dials or other suitable pieces of apparatus.

The door 114 closes against the main body 112 to define a recess 118 there-between. A dialysis fluid mixing and pumping cartridge 130 may be housed in the recess 118 as disclosed in WO2006120415, WO2015022537, WO2018/178651, the contents of which are expressly incorporated herein by reference. The main body 112 has a platen 120 behind which is an engine portion (not shown for clarity). The platen 120 is configured to receive the dialysis fluid mixing and pumping cartridge 130 within the recess 118. The engine portion includes a pneumatic pump for providing pressure and vacuum to operate the dialysis machine 100.

The dialysis machine 100 is further provided with a dialysis machine controller 150.

The liquid sanitizer 200 is provided within the dialysis machine 100. The liquid sanitizer 200 is provided within the main body 112 of the dialysis machine 100. A dialysis machine fluid circuit 140 is fluidly connected to the liquid sanitizer 200 as will be described in more detail below.

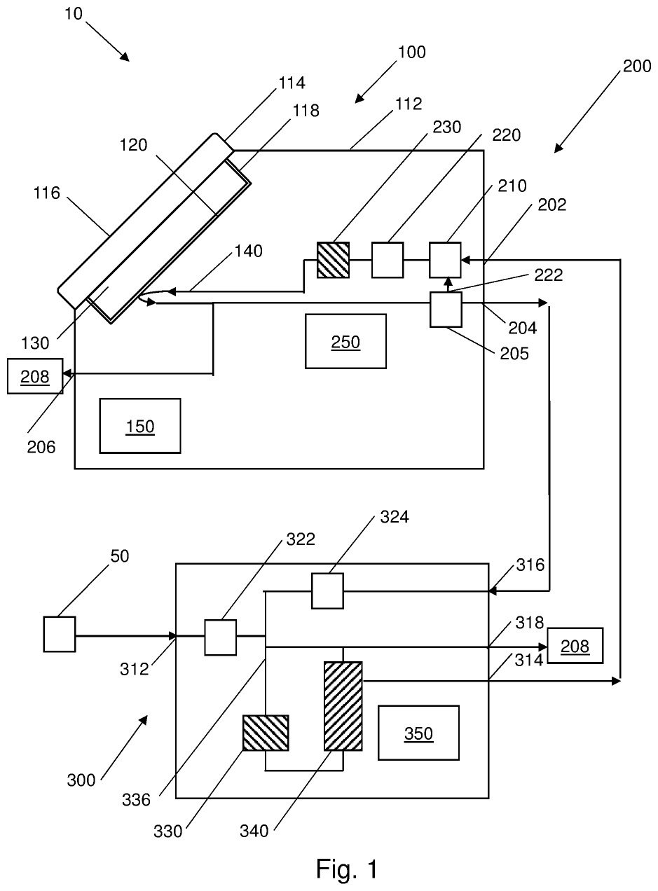

The dialysis machine has a network of fluid pathways generally designated fluid circuit 140. In dialysis use—the dialysis treatment mode—the dialysis machine fluid circuit 140 is connectable to the dialysis fluid mixing and pumping cartridge 130, where dialysis water heated to approximately 37 degrees Celsius is drawn off. In sanitization use—liquid sanitization mode—the dialysis machine fluid circuit 140 is not connected to the dialysis fluid mixing and pumping cartridge 130, instead dialysis water heated to approximately 80 to 85 degrees Celsius is not drawn off to the dialysis fluid mixing and pumping cartridge 130 but follows the arrows on FIG. 1 along the dialysis machine fluid circuit 140.

The liquid sanitizer 200 has a number of fluid connections comprising a liquid sanitizer inlet 202, a liquid sanitizer outlet 204, a liquid sanitizer drain port 206 and a liquid sanitizer return line 222. The liquid sanitizer drain port 206 is fluidly connected to a drain 208.

The liquid sanitizer 200 has an inlet water temperature sensor 210, a liquid sanitizer tank 220, a liquid sanitizer pump 230, and a liquid sanitizer controller 250. The liquid sanitizer pump 230 may be a de-aeration pump, or another form of suitable pump. The liquid sanitizer pump 230 is disposed downstream of the liquid sanitizer tank 220 and inlet water temperature sensor 210. The liquid sanitizer tank 220 has a liquid sanitizer heater 240.

The liquid sanitizer inlet 202 is fluidly connected to the liquid sanitizer tank 220 with the inlet water temperature sensor 210 adjacent the liquid sanitizer inlet 202, upstream of the liquid sanitizer tank 220. The liquid sanitizer pump 230 is fluidly connected to the liquid sanitizer tank 220. The dialysis machine fluid circuit 140 is fluidly connected to the liquid sanitizer pump 230. The dialysis machine fluid circuit 140 is fluidly connected to the liquid sanitizer outlet 204 and separately, to the inlet water temperature sensor 210 via a liquid sanitizer return line 222. A liquid sanitizer outlet valve 205 positioned adjacent the liquid sanitizer outlet 204 controls the flow via either the liquid sanitizer outlet 204 or the liquid sanitizer return line 222.

Thus within the liquid sanitization system 10 a closed fluid circuit is provided comprising the inlet water temperature sensor 210, the liquid sanitizer tank 220, the liquid sanitizer pump 230, the dialysis machine fluid circuit 140 and the liquid sanitizer return line 222. This is the first closed fluid circuit.

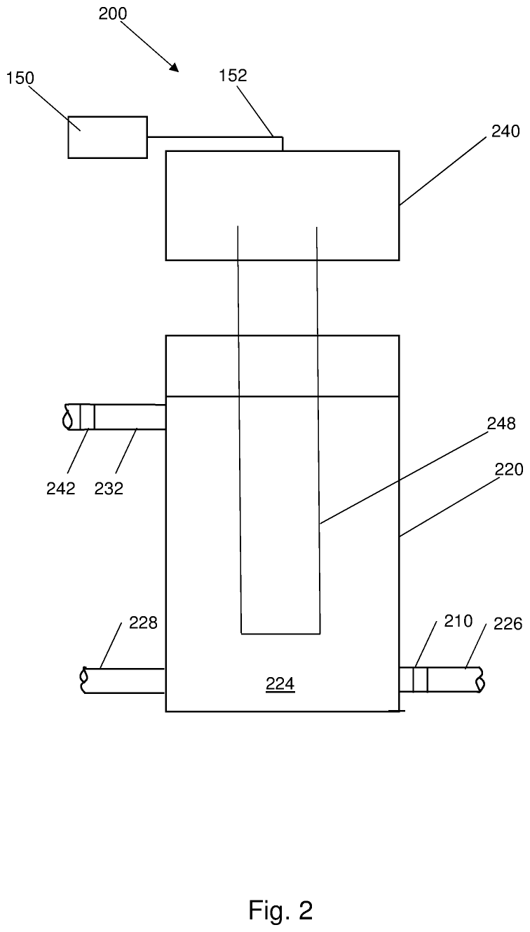

The detailed structure of the liquid sanitizer 200 is shown in FIG. 2. The liquid sanitizer tank 220 contains, in use, a volume of water 224. The liquid sanitizer tank 220 has a tank inlet 226, a tank drain 228 and a tank outlet 232. The tank inlet 226 is fluidly connectable to a water source (the reverse osmosis machine). The tank drain 228 is fluidly connectable to the drain 208. The tank outlet 232 is fluidly connected to the liquid sanitizer pump 230.

The liquid sanitizer heater 240 has a heating element 248 arranged to heat the volume of water 224 contained within the liquid sanitizer tank 220, in this case by immersion in the volume of water 224. The liquid sanitizer heater 240 is electronically connected to the dialysis machine controller 150 by a heater connector 152.

Temperature sensors are arranged in the liquid sanitizer 200. An outlet temperature sensor 242 is arranged on the tank outlet 232 adjacent the liquid sanitizer tank 220. The inlet water temperature sensor 210 is arranged on the tank inlet 226 adjacent the liquid sanitizer tank 220. The temperature sensors 242, 210 are electronically connected to the dialysis machine controller 150 via sensor connectors. The connectors may be wired or wireless. The dialysis machine controller 150 may be remote to both the liquid sanitizer tank 220 and liquid sanitizer heater 240. The dialysis machine controller 150 thereby controls both the heating of the water and receives the temperature values for the sanitizing water circuit.

Referring back to FIG. 1, the reverse osmosis (RO) machine 300 has an RO pump 330, an RO filter membrane 340 and an RO controller 350.

The RO controller 350 is connected to the dialysis machine controller 150. The dialysis machine controller 150 can communicate with and control the RO controller 350.

The reverse osmosis (RO) machine 300 has a number of fluid connections comprising an RO inlet 312, an RO outlet 314, an RO return 316, an RO drain 318. The RO inlet 312 is connected to a water source 50, such as a domestic tap, via a prefiltration stage.

The RO inlet 312 is fluidly connected to the RO pump 330 with an RO non-return valve (NRV) 322 adjacent the RO inlet 312. A common fluid line 336 fluidly connects the RO inlet 312 to the RO pump 330 downstream of the RO inlet NRV 322. The RO pump 330 is fluidly connected to the RO filter membrane 340. The RO filter membrane 340 is fluidly connected to the RO outlet 314, and separately to the RO drain.

The RO return 316 is fluidly connected to the RO pump 330, with a RO return NRV 324 adjacent the RO return 316. The common fluid line 336 fluidly connects the RO return 316 to the RO pump 330 downstream of the RO return NRV 324.

The RO drain 318 is fluidly connected to the common fluid line 336, downstream of both the RO inlet NRV 322 and RO return NRV 324.

### Liquid Sanitization System

The RO machine 300 is fluidly connected to a dialysis machine 100 and liquid sanitiser 200 as shown in FIG. 1. The RO outlet 314 is fluidly connected to the liquid sanitizer inlet 202. The RO return 316 is fluidly connected to the liquid sanitizer outlet 204.

The fluid connections are made by fluid lines shown schematically in FIG. 1. The fluid lines may be made of medical grade plastic or other suitable material. The fluid lines may be provided with thermally insulating covers which prevent thermal losses due to heat conduction.

Thus within the liquid sanitization system 10 a further closed circuit is provided comprising the inlet water temperature sensor 210, the liquid sanitizer tank 220, the liquid sanitizer pump 230, the dialysis machine fluid circuit 140, the RO return 316, the RO return NRV 324, the RO pump 330, the RO filter membrane 340 and the RO outlet 314. This is the second closed fluid circuit.

### General Setup

Depending upon the treatment setup, the dialysis machine 100 may be setup alone, or as part of a dialysis system liquid sanitization system 10 comprising the dialysis machine 100 and the water purification system. A portable unit may be used, as described in GB2006488.7, the entire contents of each are expressly incorporated herein by reference. The water purification system is removably provided within the portable unit, whereas the dialysis machine 100 is removably provided on an upper surface of the portable unit.

### Dialysis Treatment Mode

The dialysis machine 100 is used in a treatment mode to perform dialysis on a patient in a treatment session. The dialysis fluid mixing and pumping cartridge 130 is used to mix dialysis fluid constituent parts together with dialysis water from the RO machine 300, and supply the mixed dialysis fluid to a dialyser in specific quantities at specific flow rates.

The RO machine 300 in the treatment mode is operable to pump water at high pressure using the RO pump 330 (between 5 and 40 bar) across the RO filter membrane 340. This generates purified water and waste water. The purified water is pumped to the dialysis machine 100 via the RO outlet 314. The waste water is pumped to the drain 208 via the RO drain 318. The ratio of generated purified water and waste water is known as the RO recovery rate. Typically, 50% of the supplied water results in dialysis water and 50% of the supplied water results in waste water.

### Liquid Sanitization Mode

In between treatment sessions, it is necessary to sanitize the fluid connections of the dialysis machine 100 and the RO machine 300. This can be done to the dialysis machine 100 individually or to the dialysis machine 100 and the RO machine 300 combined at the same time.

The user turns on liquid sanitization system 10 via the graphical user interface of the dialysis machine 100. The appropriate Liquid Sanitization Mode is selected and thus the appropriate fluid circuit is made available.

To select the first closed fluid circuit, liquid sanitizer outlet valve 205 diverts dialysis water along the liquid sanitizer return line 222. To select the second first closed fluid circuit, liquid sanitizer outlet valve 205 diverts dialysis water out of liquid sanitizer outlet 204.

The appropriate fluid circuit is primed with dialysis water from the RO machine 300, which includes filling the liquid sanitizer tank 220 of the liquid sanitizer 200.

For the first closed fluid circuit the liquid sanitizer pump 230 is activated to pump the dialysis water around the first closed fluid circuit. The dialysis water is gradually heated from a typical initial temperature of 10 degrees Celsius to a target temperature of 80 to 85 degrees Celsius as it passes through the liquid sanitizer tank 220.

For the second closed fluid circuit the liquid sanitizer pump 230 and the RO pump 330 are activated to pump the dialysis water around the second closed fluid circuit. The RO machine controller 350 is controlled by the dialysis machine controller 150 in order to activate the RO pump 330. The dialysis water is gradually heated from a typical initial temperature of 10 degrees Celsius to a target temperature of 80 to 85 degrees Celsius as it passes through the liquid sanitizer tank 220.

Thus in both cases, the dialysis machine controller 150 activates the liquid sanitizer heater 240 to heat the volume of water 224 passing through the liquid sanitizer tank 220 via the heating element 248.

When sanitizing the dialysis machine only (first closed fluid circuit), the heated water is circulated around from the liquid sanitizer tank 220, the liquid sanitizer pump 230, the dialysis machine fluid circuit 140, the liquid sanitizer return line 222 and back to the liquid sanitizer tank 220 past inlet water temperature sensor 210.

When sanitizing the dialysis machine and the reverse osmosis machine (second closed fluid circuit), the heated water is circulated around from the liquid sanitizer tank 220, the liquid sanitizer pump 230, the dialysis machine fluid circuit 140, the RO return 316, the RO return NRV 324, the RO pump 330, the RO filter membrane 340 and the RO outlet 314 and back to the liquid sanitizer tank 220 past inlet water temperature sensor 210.

Again, in both cases, the dialysis water passes the inlet water temperature sensor 210 before re-entering the liquid sanitizer tank 220 as the dialysis water circulates around either the first or second closed fluid circuit.

The temperature of the water exiting the liquid sanitizer tank 220 via tank outlet 232 is periodically sensed by outlet temperature sensor 242, and the temperature data is periodically sent to liquid sanitizer controller 250. The temperature of the water returning to the liquid sanitizer tank 220 via tank inlet 226 is periodically sensed by inlet water temperature sensor 210, and the temperature data is periodically sent to liquid sanitizer controller 250. The liquid sanitizer controller 250 therefore periodically receives sensed temperature data to provide a feedback loop to moderate the heating of the volume of water 224 to maintain the temperature of the volume of water 224 above a threshold temperature. The threshold temperature is typically between 55 degrees Celsius and 65 degrees Celsius. The liquid sanitizer controller 250 may also moderate the heating of the volume of water 224 to maintain the temperature of the volume of water 224 below an upper temperature. The upper temperature may be between 85 degrees Celsius and 99 degrees Celsius.

When the liquid sanitizer controller 250 receives data from the inlet water temperature sensor 210 that the volume of water 224 has exceeded the threshold temperature, the liquid sanitizer controller 250 periodically samples the temperature of the volume of water 224 via the inlet water temperature sensor 210, which theoretically represents the lowest possible temperature of the water on either of first and second closed fluid circuits.

The sampling is performed periodically at, for example, 1 second intervals. The sampling intervals may be varied as appropriate. Each sampled temperature represents a time-temperature value, which can be calculated by the liquid sanitizer controller 250. The liquid sanitizer controller 250 calculates a cumulative time-temperature value for the volume of water 224 by summing the sampled time-temperature values. This is compared to a target total time-temperature value indicative of a sanitizing dose for either the first and second closed fluid circuits as appropriate.

Once the calculated cumulative time-temperature value and the target cumulative time-temperature value are equal, the liquid sanitizer controller 250 sends an output signal to indicate that a sanitizing dose has been reached. The output signal is received by the liquid sanitizer heater 240 and automatically switches off the liquid sanitizer heater 240. The output signal is received by the liquid sanitizer pump 230 which is automatically switched off. In the case of the second closed fluid circuit, the output signal is also received by the RO controller 350 which relays the signal to the RO pump 330 which is automatically switched off.

In an alternate embodiment, the liquid sanitizer controller 250 may switch off the liquid sanitizer heater 240 in advance of a sanitizing dose being reached, by calculating that there is sufficient thermal energy contained within the appropriate closed fluid circuit that the water temperature will remain above the threshold temperature for long enough to ensure a sanitizing dose is reached. In that case, periodic sampling would be continued, such that the liquid sanitizer controller 250 is able to send the output signal to indicate that a sanitizing dose had indeed been reached.

The output signal is received by the graphical user interface, which displays the text “COMPLETE” in reference to the completed sanitizing dose. In alternate embodiments, the graphical user interface includes an audible alarm. The audible alarm can be configured to bleep repeatedly until the liquid sanitization system 10 is turned off.

In alternate embodiments the liquid sanitizer controller 250 calculates two cumulative time-temperature values and two target total time-temperature value indicative of a sanitizing dose. A first cumulative time-temperature value for the first closed fluid circuit and a first target time-temperature value indicative of a sanitizing dose in the first closed fluid circuit. A second cumulative time-temperature value for the second closed fluid circuit and a second target time-temperature value indicative of a sanitizing dose in the second closed fluid circuit. Having two separate cumulative time-temperature values and target time-temperature values allows for optimised control of the sanitization of the RO and dialysis machine. The sanitization can be tailored to each component using the different sanitization target times.

The heat sanitization processes may be achieved by means of the AO method which uses a knowledge of the lethality to biofilms of the particular process at different temperatures to assess the overall lethality to biofilms of the cycle and express this as the equivalent exposure time at a specified temperature.

The A value is a measure of the heat resistance of a microorganism. A is defined as the equivalent time in seconds at 80° C. to give a sanitization effect. The z value indicates the temperature sensitivity of the reaction. It is defined as the change in temperature required to change the A value by a factor of 10. When the z value is 10° C., the term A0 is used. The A0 value of moist heat sanitization process is the equivalent time in seconds at a temperature of 80° C. delivered by that process to the product with reference to microorganisms possessing a z value of 10° C.

$${A}_{0} = Σ10 {e}^{\frac{T - 80}{z}} dt$$

A0 is the A value when z is 10° C.;
t is the chosen time interval, in seconds;
and is the temperature in the load in ° C.

In calculating A0 values a temperature threshold for the integration is set at 65° C. since for temperatures below 65° C. the z and D value of thermophilic organisms may change dramatically and below 55° C. there are a number or organisms which will actively replicate. In dialysis current practice, raising the temperature to 80° C. for 30 minutes gives a benchmark value A0 equal to 1800.

The A0 value for the first fluid circuit comprising the dialysis machine is equal to A0₁. The A0 value for the second fluid circuit comprising the water purification system is equal to A0₂. A0₂ is greater than A0₁.

### List of Reference Numerals

liquid sanitization system 10

water source 50

dialysis machine 100

main body 112

door 114

Liquid Crystal Display (LCD) unit 116

recess 118

platen 120

dialysis fluid mixing and pumping cartridge 130

dialysis machine fluid circuit 140

dialysis machine controller 150

heater connector 152

liquid sanitizer 200

liquid sanitizer inlet 202

liquid sanitizer outlet 204

liquid sanitizer outlet valve 205

liquid sanitizer drain port 206

drain 208

inlet water temperature sensor 210

liquid sanitizer tank 220

liquid sanitizer return line 222

volume of water 224

tank inlet 226

tank drain 228

liquid sanitizer pump 230

tank outlet 232

liquid sanitizer heater 240

outlet temperature sensor 242

heating element 248

liquid sanitizer controller 250

reverse osmosis (RO) machine 300

RO inlet 312

RO outlet 314

RO return 316

RO drain 318

RO non-return valve (NRV) 322

RO return NRV 324

RO pump 330

common fluid line 336

RO filter membrane 340

RO controller 350

1-18. (canceled)

19. A dialysis system comprising:
a dialysis machine having a main body portion;
a water purification system, the water purification system being separate to the dialysis machine; and
a liquid sanitizer;
wherein:
the liquid sanitizer is provided within the main body portion of the dialysis machine;
the liquid sanitizer is fluidly connected between the dialysis machine and the water purification system,
the liquid sanitizer having a heater arranged to heat a volume of liquid, a temperature sensor arranged to sense the temperature of the volume of liquid and a liquid sanitizer controller,
the dialysis system defining a first closed fluid circuit comprising the dialysis machine and the liquid sanitizer, wherein the first closed fluid circuit is wholly within the dialysis machine main body portion, and a second closed fluid circuit comprising the water purification system, the dialysis machine and the liquid sanitizer; and
the liquid sanitizer is configured to effect sanitization of the first closed fluid circuit and the second closed fluid circuit.

20. The dialysis system of claim 19, wherein the liquid sanitizer controller is controlled by a dialysis machine controller.

21. The dialysis system of claim 20, wherein the dialysis machine is provided with a graphical user interface, wherein the graphical user interface can provide instructions to the dialysis machine controller.

22. The dialysis system of claim 19, wherein the water purification system comprises a reverse osmosis (RO) machine.

23. The dialysis system of claim 24, wherein the reverse osmosis machine has a RO machine controller.

24. The dialysis system of claim 19, wherein the liquid sanitizer controller is configured to determine a time-temperature value for the volume of liquid periodically once a threshold temperature has been exceeded and calculate a cumulative time-temperature value for the first closed fluid circuit and the second closed fluid circuit.

25. The dialysis system of claim 19, wherein the liquid sanitizer temperature sensor is an inlet water temperature sensor arranged on a tank inlet adjacent a liquid sanitizer tank.

26. The dialysis system of claim 19, wherein a liquid sanitizer outlet valve is positioned adjacent a liquid sanitizer outlet to control the flow via either the liquid sanitizer outlet or a liquid sanitizer return line.

27. The dialysis system of claim 23, wherein the RO machine controller is controlled by the dialysis machine controller.

28. A method of heat sanitization of a dialysis system, the method comprising the steps of:
providing a dialysis machine having a main body portion, a water purification system, the water purification system being separate to the dialysis machine, and a liquid sanitizer, wherein the liquid sanitizer is provided within the main body portion of the dialysis machine;
wherein:
the liquid sanitizer being fluidly connected between the dialysis machine and the water purification system, and
the liquid sanitizer having a heater arranged to heat a volume of liquid and a temperature sensor arranged to sense the temperature of the volume of liquid;
heating the volume of liquid from an initial temperature to exceed a threshold temperature, maintaining the volume of water above the threshold temperature;
circulating the volume of liquid through a first closed fluid circuit comprising the dialysis machine and the liquid sanitizer; and
circulating the volume of liquid through a second closed fluid circuit comprising the water purification system, the dialysis machine and the liquid sanitizer to effect a sanitizing dose in the first fluid circuit and the second fluid circuit.

29. The method according to claim 28, wherein the steps of circulating the volume of liquid through the first closed fluid circuit and circulating the volume of liquid through the second closed fluid circuit are terminated in a sequential fashion.

30. The method according to claim 28, further comprising:
determining a time-temperature value for the volume of liquid periodically once the threshold temperature has been exceeded and calculating a cumulative time-temperature value based upon the determined time-temperature value.

31. The method according to claim 30, wherein the cumulative time-temperature value is calculated according to
$${A}_{0} = Σ10 {e}^{\frac{T - 80}{z}} dt$$
A₀ is the A value when z is 10° C.;
t is the chosen time interval, in seconds;
and is the temperature in the load in ° C.

32. The method according to claim 31, where the A₀ value for the first closed fluid circuit is equal to A_{O1} and the A₀ value for the second closed fluid circuit is equal to A_{O2}, where A_{O2} is greater than A_{O1}.

33. The method according to claim 28, wherein the step of circulating the volume of liquid through a second closed fluid circuit includes using a water purification system pump.

34. The method according to claim 30, further comprising providing an output signal once the cumulative time-temperature value has reached a level indicative of a sanitizing dose in both the first fluid circuit and the second closed fluid circuit.

35. The method according to claim 30, further comprising ceasing circulation through the second closed fluid circuit once the cumulative time-temperature value equals a target cumulative time-temperature value.

36. The method according to claim 35, further comprising setting at least one of the threshold temperature, the upper temperature or the target cumulative time-temperature value.
